(19) United States
(12) Patent Application Publication
(10) Pub. No.: US 2005/0112633 A1
(43) Pub. Date: May 26, 2005

(54) Alternatively spliced isoforms of sodium channel, voltage gated, type VIII, alpha (SCN8A)

(76) Inventors: Christopher D. Armour, Kirkland, WA (US); John C. Castle, Seattle, WA (US); Philip W. Garrett-Engele, Seattle, WA (US); Zhengyan Kan, Bellevue, WA (US); Patrick M. Loerch, Boston, MA (US); Nicholas F. Tsinoremas, Sammamish, WA (US)

Correspondence Address: R. Douglas Bradley;Rosetta Inpharmatics LLC, Legal Department, 401 Terry Avenue North, Seattle, WA, 98109, US

(21) Appl. No.: 10/942,522

(22) Filed: Sep. 16, 2004

Related U.S. Application Data
(60) Provisional application No. 60/503,694, filed on Sep. 17, 2003.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention features nucleic acids and polypeptides encoding three novel splice variant isoforms of sodium channel, voltage gated, type VIII, alpha (SCN8A). The polynucleotide sequences of SCN8Asv1.1, SCN8Asv1.2 and SCN8Asv2 are provided by SEQ ID NO 3, SEQ ID NO 5, and SEQ ID NO 7, respectively. The amino acid sequences for SCN8Asv1.1, SCN8Asv1.2, and SCN8Asv2 are provided by SEQ ID NO 4, SEQ ID NO 6, and SEQ ID NO 8, respectively. The present invention also provides methods for using SCN8Asv1.1, SCN8Asv1.2, and SCN8Asv2 polynucleotides and proteins to screen for compounds that bind to SCN8Asv1.1, SCN8Asv1.2, and SCN8Asv2, respectively.

This application claims priority to U.S. Provisional Patent Application Ser. No. 60/503,694 filed on Sep. 17, 2003, which is incorporated by reference herein in its entirety.

### BACKGROUND OF THE INVENTION

The references cited herein are not admitted to be prior art to the claimed invention.

Human and mouse genomes contain at least ten related sodium channel genes encoding proteins having highly conserved sequences (reviewed in Meisler et al., 2001, Neuroscientist 7(2): 136-145; Wood and Baker, 2001, Curr. Opin. in Pharm. 1: 17-21; Goldin et al., 2000, Neuron 28: 365-368). Sodium channels mediate the influx of sodium ions in response to changes in membrane potential in electrically excitable cells such as neurons and muscle (Catterall, W. A., 1992, Physiol. Rev. 72, (Suppl.) S 15-S48). Sodium channels consist of an about 260 kilo Dalton (kD) pore-forming alpha (α) subunit and auxiliary beta (β) subunits that modify the kinetics of voltage dependent channel gating (reviewed in Catterall, W. A., 2000, Neuron 26: 13-25; Goldin et al., 2000; Wood and Baker, 2001). The alpha subunit of a voltage gated sodium channel has four homologous domains (Domains I, II, III and IV), each containing six α-helical transmembrane segments (reviewed in Catterall, W. A., 2000; Goldin et al., 2000; Wood and Baker, 2001). While the β-subunits are required for normal kinetics and gating of the channel, expression of the α-subunit is alone sufficient for sodium currents in Xenopus oocytes (Noda et al., 1986, Nature 322: 213-216; Akopian et al., 1996, Nature 379: 257-262; reviewed in Catterall, W. A., 2000).

Although sodium channels have highly conserved protein sequences, they have diverged with respect to tissue-specific and temporal gene expression. Four genes—SCN1A (Naᵥ1.1), SCN2A (Naᵥ1.2), SCN3A (Naᵥ1.3), and SCN8A (Naᵥ1.6)—are highly expressed in the central nervous system. Three genes—SCN9A (Naᵥ1.7), SCN10A (Naᵥ1.8), and SCN11A (Naᵥ1.9)—are predominantly expressed in the peripheral nervous system. And three genes—SCN4A (Naᵥ1.4), SCN5A (Naᵥ1.5), and SCN7A (Naₓ)—are expressed in muscle (reviewed in Meisler et al., 2000).

Mutations in sodium channel genes have been implicated in several human disorders. Two mutations that cause generalized epilepsy with febrile seizures have been identified in the SCN1A (Naᵥ1.1) gene (Escayg et al., 2000, Nat. Genet. 24: 343-345). Brugada syndrome, characterized by ventricular fibrillation, heart failure and sudden death, is associated with mutations in the cardiac sodium channel SCN5A (Naᵥ1.5) (Chen et al., 1998, Nature 392: 293-296; Baroudi et al., 2000, FEBS Lett. 467: 12-16). Mutations in SCN5A have been associated with the cardiac arrhythmia of Long-QT syndrome type 3 (Wang et al., 1995, Cell 80: 805-811). Mutations in the skeletal muscle channel gene SCN4A (Naᵥ1.4) have been implicated in hyperkalaemic periodic paralysis (Bulman, D. E., 1997, Hum. Mol. Gen. 6: 1679-1685; Rojas et al., 1999, Am. J. Physiol. 276: C259-C266). In the mouse, mutations in SCN10A (Naᵥ1.8) have been linked to reduced pain sensitivity (Akopian et al., 1999, Nat. Neurosci. 2: 541-548; Laird et al., 2002, J. Neurosci. 22(19): 8352-8356).

Human SCN8A protein is highly conserved compared to other homologs in other species and has 98% sequence identity to mouse SCN8A (Plummer et al., 1998, Genomics 54: 287-296). This high degree of sequence identity suggests that the mouse and human proteins perform the same or very similar cellular functions. This conservation of sequence similarity also suggests that scn8a deficient mice will be useful models for human disease resulting from SCN8A mutations.

In the mouse, SCN8A is associated with inherited neurological disorders including ataxia, dystonia, severe muscle weakness, and paralysis. The scn8a(med) and scn8a(med^{J}) mutations in mice interfere with splicing and are predicted to encode non-functional proteins (Kohrman et al., 1996, J. Biol. Chem. 271: 17576-17581). The scn8a(med) mutation is caused by the insertion of a partial LINE element into exon 2 of SCN8A. The med transcript is spliced from exon 1 to a cryptic acceptor site in intron 2, creating a premature stop codon in the scn8a(med) coding sequence that results in a truncated protein of 127 amino acids (Kohrman et al., 1996). A four-base pair deletion within the 5′ donor site of exon 3 in the scn8a(med^{J}) allele results in splicing from exon 1 to exon 4, creating a premature stop codon and a truncated protein of 101 amino acids. (Kohrman et al., 1996). Mice with scn8a(med) and scn8a(med^{J}) mutations exhibit severe neurological disorders including early onset progressive paralysis of the hind limbs, severe muscle atrophy and juvenile lethality (Burgess et al., 1995, Nat. Genet. 10: 461-465; Sidman et al., 1979, Ann. N.Y. Acad. Sci. 317: 497-505; reviewed in Meisler et al., 1997, Ann. Med. 29: 569-574). Muscle atrophy in these mice most likely results from a loss of functional innervation (Duchen, L. W. & Stefani, E., 1971, J. Physiol. (Lond.) 212: 535-548; Angaut-Petit et al., 1982, Proc. R. Soc. Lond. B. Biol. Sci. 215: 117-125). When these mutations are placed within a C3H genetic background instead of a C57BL/6J mouse strain background, the scn8a(med^{J}) mutation, but not the scn8a(med) mutation, causes dystonia, a neurological disorder characterized by involuntary muscle contraction that produces twisting movements and sustained abnormal postures (Sprunger et al., 1999, Hum. Mol. Genet. 8: 471-479). The difference in phenotype between the scn8a(med) and scn8a(med^{J}) mutations within the C3H genetic background has been attributed to a low level of expression of the full length SCN8A transcript in scn8a(med^{J}) mice.

Other mutations in mouse SCN8A have also been associated with neurological phenotypes. A single amino acid substitution (from threonine to alanine) in a highly conserved cytoplasmic loop between trans-membrane segments 4 and 5 in Domain III of mouse SCN8A has been implicated in cerebellar ataxia (Kohrman et al., 1996, J. Neurosci. 16: 5993-5999). Genetic complementation and transcript expression analysis suggest that the mouse dmu mutation, which causes an early-onset progressive loss of mobility of the hind limbs and subsequent lethality in the first month of life, is allelic with scn8a (Repentigny et al., 2001, Hum. Mol. Genet. 10: 1819-1827). The broad range of phenotypes observed in mice with mutations in SCN8A suggests that human SCN8A mutations will also be associated with a variety of inherited neurological disorders.

Sodium channel activity can be modified by several small molecules and peptides. The pore blockers tetrodotoxin and saxitoxin bind to sodium channels with varying affinities, depending on the amino acid sequence of these channels at key structural positions (reviewed in Catterall, W. A., 2000). For example, tetrodotoxin resistant (TTX-R) channels contain cysteine or serine residues in the SS2 segment of Domain I (Akopian, et al., 1996, Nature 379: 257-262), whereas an aromatic residue is located in tetrodotoxin sensitive (TTX-S) channels (Satin et al., 1992, Science 256: 1202-1205). Likewise, cadmium is a high-affinity blocker of cardiac sodium channels but not of brain or skeletal muscle sodium channels due to amino acid variations in these channels in Domain I (Backx et al., 1992, Science 257: 248-251; Satin et al., 1992, Science 256: 1202-1205). Sodium channel blocking drugs of diverse structure have been used as anti-arrhythmic drugs and anti-convulsants (reviewed in Catterall, W. A., 2000). Peptides with sequences corresponding to the intracellular loop connecting Domains III and IV of the sodium channel α subunit can also be used as pore blockers and can restore activation to sodium channels that are defective in inactivation (Eaholtz et al., 1994, Neuron 12: 1041-1048). Other neurotoxins such as α-scorpion toxins and sea anemone toxins uncouple sodium channel activation from inactivation by preventing the normal gating movement of the extracellular end of the IVS4 segment (Rogers et al., 1996, J. Biol. Chem. 271: 15950-15962; Sheets et al., 1999, Biophys. J. 77: 747-757).

Significantly, cockroach SCN8A splice variants differ from one another with respect to their gating properties and sensitivity to deltamethrin, a pyrethroid insecticide (Tan, et al., 2002, J. Neurosci. 22(13): 5300-5309). As these examples illustrate, the effectiveness of small molecule effectors on sodium channels is largely dependent on the specific amino acid sequence and structure of the sodium channel. It is therefore likely that these small molecules will modulate the activity of splice variants with alternative structures and sequences differently than the reference protein.

Consistent with scn8a mice exhibiting neurological defects, SCN8A is expressed in neurons throughout the central and peripheral nervous systems (Burgess et al., 1995; Schaller et al., 1995, J. Neurosci. 15: 3231-3242; Felts et al., 1997, Brain Res. Mol. Brain Res. 45: 71-82; Dietrich et al., 1998, J. Neurochem. 70: 2262-2272). SCN8A transcripts are also developmentally regulated in the mouse. SCN8A transcripts are present at low levels in brain and spinal cord before birth and increase to adult levels by three weeks of age. (Plummer et al., 1997, J. Biol. Chem. 272: 24008-24015). Furthermore, expression of SCN8A splice variants is differentially regulated in a temporal and tissue-specific manner. For example, mouse SCN8A mRNA can be found with exon 18N, an alternative exon 18 called exon 18A, or no exon 18 (Δ18) (Plummer et al., 1997). Exon 18N, but not exon 18A, has a premature stop codon that results in translation of a truncated protein (Plummer et al., 1997). While there is a substantial increase in expression of SCN8A mRNA with exon 18A after birth, expression of SCN8A mRNA with exon 18N or exon Δ18 decreases (Plummer et al., 1997). Furthermore, the major transcript of SCN8A in brain and spinal cord contains exon 18A, while exon 18N or exon Δ18 containing transcripts predominate in all non-neuronal tissues tested (Plummer et al., 1997). Truncated SCN8A which results from a transcript containing exon 18N may have some function in non-neuronal cells. Alternatively, expression of a SCN8A transcript with exon 18N may act as a “fail-safe” mechanism that produces a non-functional protein to prevent inappropriate expression of an active full-length protein (Plummer et al., 1997). The developmental and tissue-specific regulation of the exon 18 region of SCN8A transcripts illustrates the importance of identifying and analyzing splice variants of SCN8A in order to understand the function and regulation of SCN8A.

Because of the multiple therapeutic values of drugs targeting sodium channels, including SCN8A, there is a need in the art for compounds that selectively bind to isoforms of SCN8A. The present invention is directed towards two novel SCN8A isoforms (SCN8Asv1 and SCN8Asv2) and uses thereof.

### BRIEF DESCRIPTION OF THE FIGURES

FIG. 1A illustrates the exon structure of human SCN8A mRNA corresponding to the known long reference form of SCN8A mRNA (labeled NM_{—}014191.1) and the exon structure corresponding to the inventive short form splice variant transcripts (labeled SCN8Asv1 and SCN8Asv2). FIG. 1B depicts the nucleotide sequences of the exon junctions resulting from the splicing of exon 2 to exon 7 (SEQ ID NO 1) in the case of SCN8Asv1 mRNA and the splicing of exon 5 to exon 6A (SEQ ID NO 2) in the case of SCN8Asv2 mRNA. In FIG. 1B, SEQ ID NO 1, the nucleotides shown in italics represent the 20 nucleotides at the 3′ end of exon 2 and the nucleotides shown in underline represent the 20 nucleotides at the 5′ end of exon 7; and in the case of SEQ ID NO 2, the nucleotides shown in italics represent the 20 nucleotides at the 3′ end of exon 5 and the nucleotides shown in underline represent the 20 nucleotides at the 5′ end of exon 6A.

### SUMMARY OF THE INVENTION

Microarray experiments, RT-PCR, and DNA sequence analysis have been used to identify and confirm the presence of novel splice variants of human SCN8A mRNA. More specifically, the present invention features polynucleotides encoding different protein isoforms of SCN8A. A polynucleotide sequence encoding SCN8Asv1.1 is provided by SEQ ID NO 3. An amino acid sequence for SCN8Asv1.1 is provided by SEQ ID NO 4. A polynucleotide sequence encoding SCN8Asv1.2 is provided by SEQ ID NO 5. An amino acid sequence for SCN8Asv1.2 is provided by SEQ ID NO 6. A polynucleotide sequence encoding SCN8Asv2 is provided by SEQ ID NO 7. An amino acid sequence for SCN8Asv2 is provided by SEQ ID NO 8.

Thus, a first aspect of the present invention describes a purified SCN8Asv1.1 encoding nucleic acid, a purified SCN8Asv1.2 encoding nucleic acid, and a purified SCN8Asv2 encoding nucleic acid. The SCN8Asv1.1 encoding nucleic acid comprises SEQ ID NO 3 or the complement thereof. The SCN8Asv1.2 encoding nucleic acid comprises SEQ ID NO 5 or the complement thereof. The SCN8Asv2 encoding nucleic acid comprises SEQ ID NO 7 or the complement thereof. Reference to the presence of one region does not indicate that another region is not present. For example, in different embodiments the inventive nucleic acid can comprise, consist, or consist essentially of an encoding nucleic acid sequence of SEQ ID NO 3, can comprise, consist, or consist essentially of the nucleic acid sequence of SEQ ID NO 5, or alternatively can comprise, consist, or consist essentially of the nucleic acid sequence of SEQ ID NO 7.

Another aspect of the present invention describes a purified SCN8Asv1.1 polypeptide that can comprise, consist or consist essentially of the amino acid sequence of SEQ ID NO 4. An additional aspect describes a purified SCN8Asv1.2 polypeptide that can comprise, consist, or consist essentially of the amino acid sequence of SEQ ID NO 6. An additional aspect describes a purified SCN8Asv2 polypeptide that can comprise, consist, or consist essentially of the amino acid sequence of SEQ ID NO 8.

Another aspect of the present invention describes expression vectors. In one embodiment of the invention, the inventive expression vector comprises a nucleotide sequence encoding a polypeptide comprising, consisting, or consisting essentially of SEQ ID NO 4, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 6, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 8, wherein the nucleotide sequence is transcriptionally coupled to an exogenous promoter.

Alternatively, the nucleotide sequence comprises, consists, or consists essentially of SEQ ID NO 3, and is transcriptionally coupled to an exogenous promoter. In another embodiment, the nucleotide sequence comprises, consists, or consists essentially of SEQ ID NO 5, and is transcriptionally coupled to an exogenous promoter. In another embodiment, the nucleotide sequence comprises, consists, or consists essentially of SEQ ID NO 7, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 3, SEQ ID NO 5, or SEQ ID NO 7, or a nucleotide sequence encoding a polypeptide comprising, consisting, or consisting essentially of an amino acid sequence of SEQ ID NO 4, SEQ ID NO 6, or SEQ ID NO 8, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing SCN8Asv1.1, SCN8Asv1.2, or SCN8Asv2 polypeptide comprising SEQ ID NO 4, SEQ ID NO 6, or SEQ ID NO 8, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to SCN8Asv1.1 as compared to one or more sodium channel isoform polypeptides that are not SCN8Asv1.1. In another embodiment, a purified antibody preparation is provided comprising antibody that binds selectively to SCN8Asv1.2 as compared to one or more sodium channel isoform polypeptides that are not SCN8Asv1.2. In another embodiment, a purified antibody preparation is provided comprising antibody that binds selectively to SCN8Asv2 as compared to one or more sodium channel isoform polypeptides that are not SCN8Asv2.

Another aspect of the present invention provides a method of screening for a compound that binds to SCN8Asv1.1, SCN8Asv1.2, SCN8Asv2, or fragments thereof. In one embodiment, the method comprises the steps of: (a) expressing a polypeptide comprising the amino acid sequence of SEQ ID NO 4 or a fragment thereof from recombinant nucleic acid; (b) providing to said polypeptide a labeled SCN8A ligand that binds to said polypeptide and a test preparation comprising one or more test compounds; (c) and measuring the effect of said test preparation on binding of said test preparation to said polypeptide comprising SEQ ID NO 4. Alternatively, this method could be performed using SEQ ID NO 6 or SEQ ID NO 8, instead of SEQ ID NO 4.

In another embodiment of the method, a compound is identified that binds selectively to SCN8Asv1.1 polypeptide as compared to one or more sodium channel isoform polypeptides that are not SCN8Asv1.1. This method comprises the steps of: providing a SCN8Asv1.1 polypeptide comprising SEQ ID NO 4; providing a sodium channel isoform polypeptide that is not SCN8Asv1.1; contacting said SCN8Asv1.1 polypeptide and said sodium channel isoform polypeptide that is not SCN8Asv1.1 with a test preparation comprising one or more test compounds; and determining the binding of said test preparation to said SCN8Asv1.1 polypeptide and to said sodium channel isoform polypeptide that is not SCN8Asv1.1, wherein a test preparation that binds to said SCN8Asv1.1 polypeptide but does not bind to said sodium channel isoform polypeptide that is not SCN8Asv1.1 contains a compound that selectively binds said SCN8Asv1.1 polypeptide. Alternatively, the same method can be performed using SCN8Asv1.2 polypeptide comprising, consisting, or consisting essentially of SEQ ID NO 6. Alternatively, the same method can be performed using SCN8Asv2 polypeptide comprising, consisting, or consisting essentially of SEQ ID NO 8.

In another embodiment of the invention, a method is provided for screening for a compound able to bind to or interact with a SCN8Asv1.1 protein or a fragment thereof comprising the steps of: expressing a SCN8Asv1.1 polypeptide comprising SEQ ID NO 4 or a fragment thereof from a recombinant nucleic acid; providing to said polypeptide a labeled SCN8A ligand that binds to said polypeptide and a test preparation comprising one or more compounds; and measuring the effect of said test preparation on binding of said labeled SCN8A ligand to said polypeptide, wherein a test preparation that alters the binding of said labeled SCN8A ligand to said polypeptide contains a compound that binds to or interacts with said polypeptide. In an alternative embodiment, the method is performed using SCN8Asv1.2 polypeptide comprising, consisting, or consisting essentially of SEQ ID NO 6 or a fragment thereof. In an alternative embodiment, the method is performed using SCN8Asv2 polypeptide comprising, consisting, or consisting essentially of SEQ ID NO 8 or a fragment thereof.

Another aspect of the present invention provides a method of screening for a compound that binds to one or more sodium channel isoform polypeptides that are not SCN8Asv1.1. This method comprises the steps of: providing a SCN8Asv1.1 polypeptide comprising SEQ ID NO 4; providing a sodium channel isoform polypeptide that is not SCN8Asv1.1; contacting said SCN8Asv1.1 polypeptide and sodium channel isoform polypeptide that is not SCN8Asv1.1 with a test preparation comprising one or more test compounds; and determining the binding of said test preparation to said SCN8Asv1.1 polypeptide and to said sodium channel isoform polypeptide that is not SCN8Asv1.1, wherein a test preparation that binds to said sodium channel isoform polypeptide that is not SCN8Asv1.1 but not to said SCN8Asv1.1 polypeptide contains a compound that selectively binds said sodium channel isoform polypeptide. Alternatively, the same method can be performed using SCN8Asv1.2 polypeptide comprising, consisting, or consisting essentially of SEQ ID NO 6. Alternatively, the same method can be performed using SCN8Asv2 polypeptide comprising, consisting, or consisting essentially of SEQ ID NO 8.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “SCN8A” refers to a sodium channel, voltage gated, type VIII, alpha (NP_{—}055006). In contrast, reference to an SCN8A isoform includes NP_{—}055006 and other polypeptide isoform variants of SCN8A.

As used herein, “SCN8Asv1.1”, “SCN8Asv1.2”, and “SCN8Asv2” refer to splice variant isoforms of human SCN8A protein, wherein the splice variants have the amino acid sequence set forth in SEQ ID NO 4 (for SCN8Asv1.1), SEQ ID NO 6 (for SCN8Asv1.2), and SEQ ID NO 8 (for SCN8Asv2).

As used herein, “SCN8A” refers to polynucleotides encoding SCN8A.

As used herein, “SCN8Asv1” refers to polynucleotides that are identical to SCN8A encoding polynucleotides, except that the sequences represented by exons 3, 4, 5, and 6 of the SCN8A messenger RNA are not present in SCN8Asv1.

As used herein, “SCN8Asv1.1” refers to polynucleotides encoding SCN8Asv1.1 having an amino acid sequence set forth in SEQ ID NO 4. As used herein, “SCN8Asv1.2” refers to polynucleotides encoding SCN8Asv1.2 having an amino acid sequence set forth in SEQ ID NO 6. As used herein, “SCN8Asv2” refers to polynucleotides encoding SCN8Asv2 having an amino acid sequence set forth in SEQ ID NO 8.

As used herein, a “sodium channel isoform” is any isoform of any sodium channel from any organism, including but not limited to human SCN1A (Naᵥ1.1), SCN2A (Naᵥ1.2), SCN3A (Naᵥ1.3), SCN4A (Naᵥ1.4), SCN5A (Naᵥ1.5), SCN6A/SCN7A (Naₓ), SCN8A (Naᵥ1.6), SCN9A (Naᵥ1.7), SCN10A (Naᵥ1.8), and SCN11A (Naᵥ1.9).

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human SCN8Asv1.1, SCN8Asv1.2 and SCN8Asv2 that are alternatively spliced isoforms of SCN8A, and to the amino acid sequences encoding these proteins. SEQ ID NO 3, SEQ ID NO 5 and SEQ ID NO 7 are polynucleotide sequences representing exemplary open reading frames that encode the SCN8Asv1.1, SCN8Asv1.2, and SCN8Asv2 proteins, respectively. SEQ ID NO 4 shows the polypeptide sequence of SCN8Asv1.1. SEQ ID NO 6 shows the polypeptide sequence of SCN8Asv1.2. SEQ ID NO 8 shows the polypeptide sequence of SCN8Asv2.

SCN8Asv1.1, SCN8Asv1.2 and SCN8Asv2 polynucleotide sequences encoding SCN8Asv1.1, SCN8Asv1.2 and SCN8Asv2 proteins, as exemplified and enabled herein include a number of specific, substantial and credible utilities. For example, SCN8Asv1.1, SCN8Asv1.2 and SCN8Asv2 encoding nucleic acids were identified in an mRNA sample obtained from a human source (see Example 1). Such nucleic acids can be used as hybridization probes to distinguish between cells that produce SCN8Asv1.1, SCN8Asv1.2, and SCN8Asv2 transcripts from human or non-human cells (including bacteria) that do not produce such transcripts. Similarly, antibodies specific for SCN8Asv1.1, SCN8Asv1.2 or SCN8Asv2 can be used to distinguish between cells that express SCN8Asv1.1, SCN8Asv1.2 or SCN8Asv2 from human or non-human cells (including bacteria) that do not express SCN8Asv1.1, SCN8Asv1.2, or SCN8Asv2.

The importance of SCN8A as a drug target for a range of neurological disorders including ataxia, dystonia, severe muscle weakness, and paralysis is evidenced by the presence of these neurological phenotypes in mice with mutations in SCN8A (Burgess et al., 1995; Sidman et al., 1995; Kohrman et al., 1996; reviewed in Meisler et al., 1997; Sprunger et al., 1999; Repentigny et al., 2001). Given the potential importance of SCN8A activity to the therapeutic management of a wide array of diseases, it is of value to identify SCN8A isoforms and identify SCN8A-ligand compounds that are isoform specific, as well as compounds that are effective ligands for two or more different SCN8A isoforms or sodium channel isoforms. In particular, it may be important to identify compounds that are effective inhibitors of a specific SCN8A isoform activity, yet do not bind to or interact with a plurality of different SCN8A isoforms or sodium channel isoforms. Compounds that bind to or interact with multiple SCN8A isoforms may require higher drug doses to saturate multiple SCN8A-isoform binding sites and thereby result in a greater likelihood of secondary non-therapeutic side effects. Furthermore, biological effects could also be caused by the interaction of a drug with the SCN8Asv1.1, SCN8Asv1.2, or SCN8Asv2 isoforms specifically. For the foregoing reasons, SCN8Asv1.1, SCN8Asv1.2, and SCN8Asv2 proteins represent useful compound binding targets and have utility in the identification of new SCN8A-ligands and sodium channel isoform-ligands exhibiting a preferred specificity profile and having greater efficacy for their intended use.

In some embodiments, SCN8Asv1.1, SCN8Asv1.2, and SCN8Asv2 activity is modulated by a ligand compound to achieve one or more of the following: prevent or reduce the risk of occurrence, or recurrence of neurological disorders including ataxia, dystonia, severe muscle weakness, and paralysis.

Compounds modulating SCN8Asv1.1, SCN8Asv1.2, or SCN8Asv2 include agonists, antagonists, and allosteric modulators. While not wishing to be limited to any particular theory of therapeutic efficacy, generally, but not always, SCN8Asv1.1, SCN8Asv1.2, or SCN8Asv2 compounds will be used to modulate the activity of the SCN8A voltage gated sodium channel. These compounds may act as pore blockers and inhibit the passage of sodium ions across the cellular membrane. Compounds that uncouple sodium channel activation from inactivation by preventing the normal gating movement of the extracellular end of the IVS4 segment may also be utilized (Rogers et al., 1996; Sheets et al., 1999). Peptides with sequences corresponding to the intracellular loop connecting Domains III and IV may act as pore blockers and may restore activation to sodium channels that are defective in inactivation (Eaholtz et al., 1994). Sodium channel blocking drugs have been used as anti-arrhythmic drugs and anti-convulsants (reviewed in Catterall, W. A., 2000). Therefore, agents that modulate SCN8A activity may be used to achieve a therapeutic benefit for any disease or condition due to, or exacerbated by, SCN8A ion channel activity.

SCN8Asv1.1, SCN8Asv1.2, or SCN8Asv2 activity can also be affected by modulating the cellular abundance of transcripts encoding SCN8Asv1.1, SCN8Asv1.2, or SCN8Asv2, respectively. Compounds modulating the abundance of transcripts encoding SCN8Asv1.1, SCN8Asv1.2, or SCN8Asv2 include a cloned polynucleotide encoding SCN8Asv1.1, SCN8Asv1.2, or SCN8Asv2, respectively, that can express SCN8Asv1.1, SCN8Asv1.2, or SCN8Asv2 in vivo, antisense nucleic acids targeted to SCN8Asv1.1, SCN8Asv1.2, or SCN8Asv2 transcripts, enzymatic nucleic acids, such as ribozymes, and RNAi nucleic acids, such as shRNAs or siRNAs, targeted to SCN8Asv1.1, SCN8Asv1.2, or SCN8Asv2 transcripts.

In some embodiments, SCN8Asv1.1, SCN8Asv1.2, or SCN8Asv2 activity is modulated to achieve a therapeutic effect upon diseases in which regulation of SCN8A is desirable. For example, neurological disorders such as ataxia, dystonia, severe muscle weakness caused by loss of muscle innervation, and paralysis may be treated by modulating SCN8Asv1.1, SCN8Asv1.2, or SCN8Asv2 sodium channel activity.

SCN8Asv1.1, SCN8Asv1.2 and SCN8Asv2 NUCLEIC ACIDS

SCN8Asv1.1 nucleic acids contain regions that encode for polypeptides comprising, consisting, or consisting essentially of SEQ ID NO 4. SCN8Asv1.2 nucleic acids contain regions that encode for polypeptides comprising, consisting, or consisting essentially of SEQ ID NO 6. SCN8Asv2 nucleic acids contain regions that encode for polypeptides comprising, consisting, or consisting essentially of SEQ ID NO 8. The SCN8Asv1.1, SCN8Asv1.2 and SCN8Asv2 nucleic acids have a variety of uses, such as use as a hybridization probe or PCR primer to identify the presence of SCN8Asv1.1, SCN8Asv1.2 or SCN8Asv2 nucleic acids, respectively; use as a hybridization probe or PCR primer to identify nucleic acids encoding for proteins related to SCN8Asv1.1, SCN8Asv1.2 or SCN8Asv2, respectively; and/or use for recombinant expression of SCN8Asv1.1, SCN8Asv1.2 or SCN8Asv2 polypeptides, respectively. In particular, SCN8Asv1.1 polynucleotides do not have the polynucleotide regions that consist of exons 3, 4, 5, and 6 of the SCN8A gene. SCN8Asv1.2 polynucleotides do not have the polynucleotide regions that consist of exons 1, 2, 3, 4, 5, and 6 of the SCN8A gene. SCN8Asv2 polynucleotides do not have the polynucleotide region that consists of exon 6 of the SCN8A gene, but has a polynucleotide region that consists of exon 6A instead of exon 6.

Regions in SCN8Asv1.1, SCN8Asv1.2, or SCN8Asv2 nucleic acid that do not encode for SCN8Asv1.1, SCN8Asv1.2, or SCN8Asv2, or are not found in SEQ ID NO 3, SEQ ID NO 5, or SEQ ID NO 7, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding SCN8Asv1.1, SCN8Asv1.2, or SCN8Asv2 related proteins from different sources. Obtaining nucleic acids encoding SCN8Asv1.1, SCN8Asv1.2, or SCN8Asv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

SCN8Asv1.1, SCN8Asv1.2, or SCN8Asv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

SCN8Asv1.1 and SCN8Asv2 Probes

Probes for SCN8Asv1.1 or SCN8Asv2 contain a region that can specifically hybridize to SCN8Asv1.1 or SCN8Asv2 target nucleic acids, respectively, under appropriate hybridization conditions and can distinguish SCN8Asv1.1 or SCN8As2 nucleic acids from each other and from non-target nucleic acids, in particular SCN8A polynucleotides containing exons 3, 4, 5, and 6. Probes for SCN8Asv1.1 or SCN8Asv2 can also contain nucleic acid regions that are not complementary to SCN8Asv1.1 or SCN8Asv2 nucleic acids.

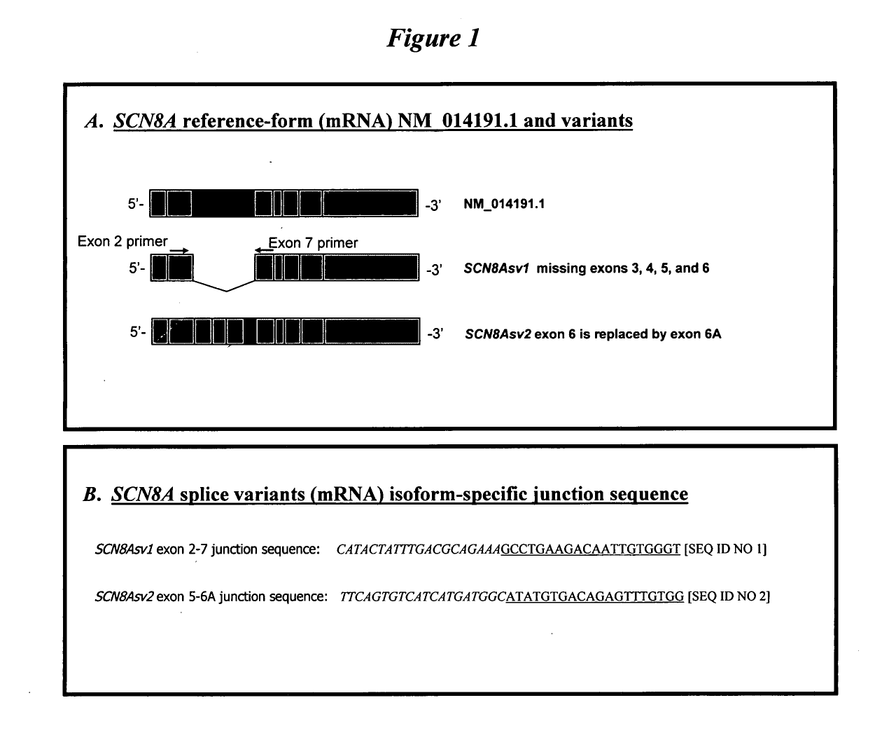

In embodiments where, for example, SCN8Asv1.1 or SCN8Asv2 polynucleotide probes are used in hybridization assays to specifically detect the presence of SCN8Asv1.1 or SCN8Asv2 polynucleotides in samples, the SCN8Asv1.1 or SCN8Asv2 polynucleotides comprise at least 20 nucleotides of the SCN8Asv1.1 or SCN8Asv2 sequence that correspond to the respective novel exon junction or novel polynucleotide regions. In particular, for detection of SCN8Asv1.1, the probe comprises at least 20 nucleotides of the SCN8Asv1.1 sequence that corresponds to an exon junction polynucleotide created by the alternative splicing of exon 2 to exon 7 of the primary transcript of the SCN8A gene (see FIGS. 1A and 1B). For example, the polynucleotide sequence: 5′ GACGCAGAAAGCCTGAAGAC 3′ [SEQ ID NO 9] represents one embodiment of such an inventive SCN8Asv1.1 polynucleotide wherein a first 10 nucleotide region is complementary and hybridizable to the 3′ end of exon 2 of the SCN8A gene and a second 10 nucleotide region is complementary and hybridizable to the 5′ end of exon 7 of the SCN8A gene (see FIG. 1B).

In another embodiment, for detection of SCN8Asv2, the probe comprises at least 20 nucleotides of the SCN8Asv2 sequence that corresponds to an exon junction polynucleotide created by the alternative splicing of exon 5 to exon 6A of the primary transcript of the SCN8A gene (see FIGS. 1A and 1B). For example, the polynucleotide sequence: 5′ TCATGATGGCA TATGTGACA 3′ [SEQ ID NO 10] represents one embodiment of such an inventive SCN8Asv2 polynucleotide wherein a first 10 nucleotide region is complementary and hybridizable to the 3′ end of exon 5 of the SCN8A gene and a second 10 nucleotide region is complementary and hybridizable to the 5′ end of exon 6A of the SCN8A gene (see FIG. 1B).

In some embodiments, the first 20 nucleotides of a SCN8Asv1.1 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 7. In some embodiments, the first 20 nucleotides of a SCN8Asv2 probe comprise a first continuous region of 5 to 15 nucleotides that is complementary and hybridizable to the 3′ end of exon 5 and a second continuous region of 5 to 15 nucleotides that is complementary and hybridizable to the 5′ end of exon 6A.

In other embodiments, the SCN8Asv1.1 or SCN8Asv2 polynucleotide comprises at least 40, 60, 80 or 100 nucleotides of the SCN8Asv1.1 or SCN8Asv2 sequence, respectively, that correspond to a junction polynucleotide region created by the alternative splicing of exon 2 to exon 7 in the case of SCN8Asv1.1, or in the case of SCN8Asv2, by the alternative splicing of exon 5 to exon 6A of the primary transcript of the SCN8A gene. In embodiments involving SCN8Asv1.1, the SCN8Asv1.1 polynucleotide is selected to comprise a first continuous region of at least 5 to 15 nucleotides that is complementary and hybridizable to the 3′ end of exon 2 and a second continuous region of at least 5 to 15 nucleotides that is complementary and hybridizable to the 5′ end of exon 7. Similarly, in embodiments involving SCN8Asv2, the SCN8Asv2 polynucleotide is selected to comprise a first continuous region of at least 5 to 15 nucleotides that is complementary and hybridizable to the 3′ end of exon 5 and a second continuous region of at least 5 to 15 nucleotides that is complementary and hybridizable to the 5′ end of exon 6A. As will be apparent to a person of skill in the art, a large number of different polynucleotide sequences from the region of the exon 2 to exon 7 splice junction and the exon 5 to exon 6A splice junction may be selected which will, under appropriate hybridization conditions, have the capacity to detectably hybridize to SCN8Asv1.1 or SCN8Asv2 polynucleotides, respectively, and yet will hybridize to a much less extent or not at all to SCN8A isoform polynucleotides wherein exon 2 is not spliced to exon 7 or wherein exon 5 is not spliced to exon 6A, respectively.

Preferably, non-complementary nucleic acid that is present has a particular purpose such as being a reporter sequence or being a capture sequence. However, additional nucleic acid need not have a particular purpose as long as the additional nucleic acid does not prevent the SCN8Asv1.1 or SCN8Asv2 nucleic acid from distinguishing between target polynucleotides, e.g., SCN8Asv1.1, or SCN8Asv2 polynucleotides, and non-target polynucleotides, including, but not limited to SCN8A polynucleotides not comprising the exon 2 to exon 7 splice junction or the exon 5 to exon 6A splice junctions found in SCN8Asv1.1 or SCN8Asv2, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a, probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

SCN8Asv1.1, SCN8Asv1.2, or SCN8Asv2 polynucleotides, such as those comprising SEQ ID NO 3, SEQ ID NO 5, or SEQ ID NO 7, respectively, can be used to make SCN8Asv1.1, SCN8Asv1.2, or SCN8Asv2 polypeptides, respectively. In particular, SCN8Asv1.1, SCN8Asv1.2, or SCN8Asv2 polypeptides can be expressed from recombinant nucleic acids in a suitable host or in vitro using a translation system. Recombinantly expressed SCN8Asv1.1, SCN8Asv1.2, or SCN8Asv2 polypeptides can be used, for example, in assays to screen for compounds that bind SCN8Asv1.1, SCN8Asv1.2, or SCN8Asv2, respectively. Alternatively, SCN8Asv1.1, SCN8Asv1.2, or SCN8Asv2 polypeptides can also be used to screen for compounds that bind to one or more SCN8A or sodium channel isoforms, but do not bind to SCN8Asv1.1, SCN8Asv1.2, or SCN8Asv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMClneo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-[(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad, Calif.).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 3, SEQ ID NO 5, or SEQ ID NO 7 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

SCN8Asv1.1. SCN8Asv1.2, and SCN8Asv2 POLYPEPTIDES

SCN8Asv1.1 polypeptides contain an amino acid sequence comprising, consisting or consisting essentially of SEQ ID NO 4. SCN8Asv1.2 polypeptides contain an amino acid sequence comprising, consisting or consisting essentially of SEQ ID NO 6. SCN8Asv2 polypeptides contain an amino acid sequence comprising, consisting or consisting essentially of SEQ ID NO 8. SCN8Asv1.1, SCN8Asv1.2, or SCN8Asv2 polypeptides have a variety of uses, such as providing a marker for the presence of SCN8Asv1.1, SCN8Asv1.2, or SCN8Asv2, respectively; use as an immunogen to produce antibodies binding to SCN8Asv1.1, SCN8Asv1.2, or SCN8Asv2, respectively; use as a target to identify compounds binding selectively to SCN8Asv1.1, SCN8Asv1.2, or SCN8Asv2, respectively; or use in an assay to identify compounds that bind to one or more SCN8A or sodium channel isoforms but do not bind to or interact with SCN8Asv1.1, SCN8Asv1.2, or SCN8Asv2, respectively.

In chimeric polypeptides containing one or more regions from SCN8Asv1.1, SCN8Asv1.2, or SCN8Asv2 and one or more regions not from SCN8Asv1.1, SCN8Asv1.2, or SCN8Asv2, respectively, the region(s) not from SCN8Asv1.1, SCN8Asv1.2, or SCN8Asv2, respectively, can be used, for example, to achieve a particular purpose or to produce a polypeptide that can substitute for SCN8Asv1.1, SCN8Asv1.2, or SCN8Asv2, or fragments thereof. Particular purposes that can be achieved using chimeric SCN8Asv1.1, SCN8Asv1.2, or SCN8Asv2 polypeptides include providing a marker for SCN8Asv1.1, SCN8Asv1.2, or SCN8Asv2 activity, respectively, and altering the activity and regulation of the SCN8A sodium channel.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional SCN8Asv1.1 SCN8Asv1.2, and SCN8Asv2

Functional SCN8Asv1.1, SCN8Asv1.2, and SCN8Asv2 are different protein isoforms of SCN8A. The identification of the amino acid and nucleic acid sequences of SCN8Asv1.1, SCN8Asv1.2, or SCN8Asv2 provide tools for obtaining functional proteins related to SCN8Asv1.1, SCN8Asv1.2, or SCN8Asv2, respectively, from other sources, for producing SCN8Asv1.1, SCN8Asv1.2, or SCN8Asv2 chimeric proteins, and for producing functional derivatives of SEQ ID NO 4, SEQ ID NO 6, or SEQ ID NO 8.

SCN8Asv1.1, SCN8Asv1.2, or SCN8Asv2 polypeptides can be readily identified and obtained based on their sequence similarity to SCN8Asv1.1 (SEQ ID NO 4), SCN8Asv1.2 (SEQ ID NO 6), or SCN8Asv2 (SEQ ID NO 8), respectively. In particular, SCN8Asv1.1 lacks the amino acids encoded by exons 3, 4, 5, and 6 of the SCN8A gene. The deletion of exons 3, 4, 5 and 6 and the splicing of exon 2 to exon 7 of the SCN8A hnRNA transcript results in a shift of the protein reading frame at the exon 2 to exon 7 splice junction, thereby creating an amino terminal peptide region that is unique to the SCN8Asv1.1 polypeptide as compared to other known SCN8A isoforms. The frame shift creates a premature termination codon four nucleotides downstream of the exon 2/exon 7 splice junction. Thus, the SCN8Asv1.1 polypeptide is also lacking the amino acids encoded by the nucleotides downstream of the premature stop codon. The SCN8Asv1.2 polypeptides lack the amino acids encoded by exons 1, 2, 3, 4, 5 and 6 of the SCN8A gene. The SCN8Asv1.2 carboxy terminal polypeptide lacks the amino acids encoded by the first 762 nucleotides of the SCN8A gene and initiates at a downstream AUG resulting in a carboxy peptide with a protein sequence that corresponds to the last 1726 amino acids of the SCN8A gene. Initiation at a downstream AUG of a bicistronic RNA is a fairly common event and can be associated with disease (Meijer and Thomas, 2002 Biochem. J., 367: 1-11; Kozak, 2002, Mammalian Genome 13:401-410). The SCN8Asv2 polypeptide lacks the amino acids encoded by exon 6 of the SCN8A gene, but has an alternative exon 6—exon 6A—in its place. Exon 6A encodes the same number of amino acids as exon 6 and the amino acid sequence of exon 6 differs from that of exon 6A at two amino acid positions (1207V and N212D with respect to the reference gene).

Both the amino acid and nucleic acid sequences of SCN8Asv1.1, SCN8Asv1.2, or SCN8Asv2 can be used to help identify and obtain SCN8Asv1.1, SCN8Asv1.2, or SCN8Asv2 polypeptides, respectively. For example, SEQ ID NO 3 can be used to produce degenerative nucleic acid probes or primers for identifying and cloning nucleic acid polynucleotides encoding for a SCN8Asv1.1 polypeptide. In addition, polynucleotides comprising, consisting, or consisting essentially of SEQ ID NO 3 or fragments thereof, can be used under conditions of moderate stringency to identify and clone nucleic acids encoding SCN8Asv1.1 polypeptides from a variety of different organisms. The same methods can also be performed with polynucleotides comprising, consisting, or consisting essentially of SEQ ID NO 5, or SEQ ID NO 7, or fragments thereof, to identify and clone nucleic acids encoding SCN8Asv1.2 and SCN8Asv2, respectively.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with SCN8Asv1.1, SCN8Asv1.2, or SCN8Asv2 obtained from a particular source, derivatives can be produced. Such derivatives include polypeptides with amino acid substitutions, additions and deletions. Changes to SCN8Asv1.1, SCN8Asv1.2, or SCN8Asv2 to produce a derivative having essentially the same properties should be made in a manner not altering the tertiary structure of SCN8Asv1.1, SCN8Asv1.2, or SCN8Asv2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

SCN8Asv1.1, SCN8Asv1.2, and SCN8Asv2 Antibodies

Antibodies recognizing SCN8Asv1.1, SCN8Asv1.2, or SCN8Asv2 can be produced using a polypeptide containing SEQ ID NO 4 in the case of SCN8Asv1.1, SEQ ID NO 6 in the case of SCN8Asv1.2, or SEQ ID NO 8 in the case of SCN8Asv2, respectively, or a fragment thereof, as an immunogen. Preferably, a SCN8Asv1.1 polypeptide used as an immunogen consists of a polypeptide of SEQ ID NO 4 or a SEQ ID NO 4 fragment having at least 10 contiguous amino acids in length corresponding to the polynucleotide region representing the junction resulting from the splicing of exon 2 to exon 7 of the SCN8A gene. Preferably, a SCN8Asv1.2 polypeptide used as an immunogen consists of a polypeptide derived from SEQ ID NO 6 or a SEQ ID NO 6 fragment, having at least 10 contiguous amino acids in length corresponding to amino acids, including and downstream of, the amino terminal initiation methionine of SCN8Asv1.2. Preferably, a SCN8Asv2 polypeptide used as an immunogen consists of a polypeptide of SEQ ID NO 8 or a SEQ ID NO 8 fragment having at least 10 contiguous amino acids in length corresponding to amino acids, including amino acids 207 and 212, which differ between exon 6 and exon 6A (1207V and N212D).

In some embodiments where, for example, SCN8Asv1.1 polypeptides are used to develop antibodies that bind specifically to SCN8Asv1.1 and not to other isoforms of SCN8A, the SCN8Asv1.1 polypeptides comprise at least 10 amino acids of the SCN8Asv1.1 polypeptide sequence corresponding to a junction polynucleotide region created by the alternative splicing of exon 2 to exon 7 of the primary transcript of the SCN8A gene (see FIG. 1). For example, the amino acid sequence: amino terminus—FDPYYLTQKA —carboxy terminus [SEQ ID NO 11] represents one embodiment of such an inventive SCN8Asv1.1 polypeptide wherein a first 9 amino acid region is encoded by nucleotide sequence at the 3′ end of exon 2 of the SCN8A gene and the last amino acid is encoded by the nucleotide sequence directly after the novel splice junction. Preferably, at least 10 amino acids of the SCN8Asv1.1 polypeptide comprise a first continuous region of 1 to 8 amino acids that is encoded by nucleotides at the 3′ end of exon 2 and a second continuous region of 1 to 8 amino acids that is encoded by nucleotides at the 5′ end of exon 7.

In other embodiments where, for example, SCN8Asv1.2 polypeptides are used to develop antibodies that bind specifically to SCN8Asv1.2 and not to other isoforms of SCN8A, the SCN8Asv1.2 polypeptides comprise at least 10 amino acids at the amino terminus of the SCN8Asv1.2 polypeptide sequence having at least 10 contiguous amino acids in length corresponding to amino acids, including and downstream of, the amino terminal initiation methionine of SCN8Asv1.2. For example, the amino acid sequence: amino terminus-MILTVFCLSV-carboxy terminus [SEQ ID NO 12], represents one embodiment of such an inventive SCN8Asv1.2 polypeptide wherein a first 10 amino acid region is encoded by a nucleotide sequence starting with the “AUG” codon 57 nucleotides downstream of the 5′ end of exon 7 of the SCN8A gene.

In other embodiments where, for example, SCN8Asv2 polypeptides are used to develop antibodies that bind specifically to SCN8Asv2 and not to other SCN8A isoforms, the SCN8Asv2 polypeptides comprise at least 10 amino acids of the SCN8Asv2 polypeptide sequence corresponding to amino acids, including amino acids 207 and 212, which differ between exon 6 and exon 6A (1207V and N212D). (see FIG. 1). For example, the amino acid sequence: amino terminus-AYVTEFVDLG-carboxy terminus [SEQ ID NO 13], represents one embodiment of such an inventive SCN8Asv2 polypeptide wherein a first 10 amino acid region is encoded by a nucleotide sequence starting with the “GCA” codon at the exon junction of exon 5 and exon 6A of the SCN8A gene. Preferably, at least 10 amino acids of the SCN8Asv2 polypeptide comprise a continuous region of 10 amino acids including amino acids 207 and 212, which differ between exon 6 and exon 6A.

In other embodiments, SCN8Asv1.1-specific antibodies are made using a SCN8Asv1.1 polypeptide that comprises at least 20, 30, 40 or 50 amino acids of the SCN8Asv1.1 sequence that corresponds to a junction polynucleotide region created by the alternative splicing of exon 2 to exon 7 of the primary transcript of the SCN8A gene. In each case the SCN8Asv1.1 polypeptides are selected to comprise a first continuous region of at least 5 to 15 amino acids that is encoded by nucleotides at the 3′ end of exon 2 and a second continuous region of 5 to 15 amino acids that is encoded by nucleotides directly after the novel splice junction.

In other embodiments, SCN8Asv1.2-specific antibodies are made using an SCN8Asv1.2 polypeptide that comprises at least 20, 30, 40, or 50 amino acids of the SCN8Asv1.2 sequence that corresponds to a polynucleotide region encoding amino acids, including and downstream of, the methionine codon located 57 nucleotides downstream of the 5′ end of exon 7 of the primary transcript of the SCN8A gene.

In other embodiments, SCN8Asv2-specific antibodies are made using a SCN8Asv2 polypeptide that comprises at least 20, 30, 40 or 50 amino acids of the SCN8Asv2 sequence that corresponds to a continuous region of amino acids including amino acids 207 and 212 of the SCN8A gene, which differ between exon 6 and exon 6A.

Antibodies to SCN8Asv1.1, SCN8Asv1.2, or SCN8Asv2 have different uses, such as to identify the presence of SCN8Asv1.1, SCN8Asv1.2, or SCN8Asv2, respectively, and to isolate SCN8Asv1.1, SCN8Asv1.2, or SCN8Asv2 polypeptides, respectively. Identifying the presence of SCN8Asv1.1 can be used, for example, to identify cells producing SCN8Asv1.1. Such identification provides an additional source of SCN8Asv1.1 and can be used to distinguish cells known to produce SCN8Asv1.1 from cells that do not produce SCN8Asv1.1. For example, antibodies to SCN8Asv1.1 can distinguish human cells expressing SCN8Asv1.1 from human cells not expressing SCN8Asv1.1 or non-human cells (including bacteria) that do not express SCN8Asv1.1. Such SCN8Asv1.1 antibodies can also be used to determine the effectiveness of SCN8Asv1.1 ligands, using techniques well known in the art, to detect and quantify changes in the protein levels of SCN8Asv1.1 in cellular extracts, and in situ immunostaining of cells and tissues. In addition, the same above-described utilities also exist for SCN8Asv1.2-specific antibodies and SCN8Asv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

SCN8Asv1.1, SCN8Asv1.2 and SCN8Asv2 Bindinz Assay

A number of compounds known to modulate sodium channel activity have been disclosed including tetrodotoxin, saxitoxin, cadmium, and other neurotoxins such as α-scorpion toxin and sea anemone toxin (Akopian et al., 1996; Satin et al., 1992; Backz et al., 1992; reviewed in Catterall, W. A., 2000). Peptide sequences corresponding to the intracellular loop connecting Domains III and IV of the sodium channel α subunit have also been reported to have efficacy as pore blockers and can restore activation to sodium channels that are defective in inactivation (Eaholtz et al., 1994). The human SCN8A reference protein (NP_{—}055006) has a tyrosine in the SS2 segment of Domain I and is therefore expected to be sensitive to tetrodotoxin. This is consistent with the finding that the SCN8A sodium channel from rat is sensitive to tetrodotoxin (Dietrich et al., 1998, J. Neurochem. 70(6): 2262-2272). Splice variants of the cockroach SCN8A sodium channel exhibit different gating properties and different sensitivities to deltamethrin, a pyrethroid insecticide (Tan et al., 2002, J. Neurosci. 22(13): 5300-5309), indicating that splice variant isoforms may have different sensitivities to ligands. Methods for expressing sodium channels in Xenopus oocytes and monitoring the activity of these channels, including analyzing the effect of compounds on the activity of sodium channel activity, have been described previously (Dietrich et al., 1998, J. Neurochem. 70(6): 2262-72; Keller et al., 2000, J. Pharmacol. Exp. Ther. 295(1): 367-72; Spampanato et al., 2001, J. Neurosci. 21(19): 7481-7490; Tan et al., 2002, J. Neurosci. 22(13): 5300-5309). Methods for screening compounds for their effects on sodium channel activity have also been disclosed (see for example U.S. 2002/0025568; U.S. 2002/0045159; WO 03/006103; Gonzales et al., 1999, Drug Discov. Today 4: 431-439). A person skilled in the art should be able to use these methods to screen SCN8Asv1.1, SCN8Asv1.2, or SCN8Asv2 polypeptides for compounds that bind to, and in some cases functionally alter, each respective SCN8A isoform protein.

SCN8Asv1.1, SCN8Asv1.2, SCN8Asv2, or fragments thereof, can be used in binding studies to identify compounds binding to or interacting with SCN8Asv1.1, SCN8Asv1.2, SCN8Asv2, or fragments thereof, respectively. In one embodiment, SCN8Asv1.1, or a fragment thereof, can be used in binding studies with a sodium channel isoform protein, or a fragment thereof, to identify compounds that: bind to or interact with SCN8Asv1.1 and other sodium channel isoforms; bind to or interact with one or more other sodium channel isoforms and not with SCN8Asv1.1; bind to or interact with SCN8Asv1.1 and not with one or more other sodium channel isoforms. A similar series of compound screens can, of course, also be performed using SCN8Asv1.2 or SCN8Asv2 rather than, or in addition to, SCN8Asv1.1. Such binding studies can be performed using different formats including competitive and non-competitive formats. Further competition studies can be carried out using additional compounds determined to bind to SCN8Asv1.1, SCN8Asv1.2, SCN8Asv2, other SCN8A isoforms, or other sodium channel isoforms.

The particular SCN8Asv1.1, SCN8Asv1.2, or SCN8Asv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using SCN8Asv1.1 expressed from a recombinant nucleic acid. Alternatively, recombinantly expressed SCN8Asv1.1 consists of the SEQ ID NO 4 amino acid sequence. In addition, binding studies are performed using SCN8Asv1.2 expressed from a recombinant nucleic acid. Alternatively, recombinantly expressed SCN8Asv1.2 consists of the SEQ ID NO 6 amino acid sequence. In addition, binding studies are performed using SCN8Asv2 expressed from a recombinant nucleic acid. Alternatively, recombinantly expressed SCN8Asv2 consists of the SEQ ID NO 8 amino acid sequence.

Binding assays can be performed using individual compounds or preparations containing different numbers of compounds. A preparation containing different numbers of compounds having the ability to bind to SCN8Asv1.1, SCN8Asv1.2, or SCN8Asv2 can be divided into smaller groups of compounds that can be tested to identify the compound(s) binding to SCN8Asv1.1, SCN8Asv1.2, or SCN8Asv2, respectively.

Binding assays can be performed using recombinantly produced SCN8Asv1.1, SCN8Asv1.2, or SCN8Asv2 present in different environments. Such environments include, for example, cell extracts and purified cell extracts containing a SCN8Asv1.1, SCN8Asv1.2, or SCN8Asv2 recombinant nucleic acid; and also include, for example, the use of a purified SCN8Asv1.1, SCN8Asv1.2, or SCN8Asv2 polypeptide produced by recombinant means which is introduced into different environments.

In one embodiment of the invention, a binding method is provided for screening for a compound able to bind selectively to SCN8Asv1.1. The method comprises the steps: providing a SCN8Asv1.1 polypeptide comprising SEQ ID NO 4; providing a sodium channel isoform polypeptide that is not SCN8Asv1.1; contacting the SCN8Asv1.1 polypeptide and the sodium channel isoform polypeptide that is not SCN8Asv1.1 with a test preparation comprising one or more test compounds; and then determining the binding of the test preparation to the SCN8Asv1.1 polypeptide and to the sodium channel isoform polypeptide that is not SCN8Asv1.1, wherein a test preparation that binds to the SCN8Asv1.1 polypeptide, but does not bind to the sodium channel isoform polypeptide that is not SCN8Asv1.1, contains one or more compounds that selectively bind to SCN8Asv1.1.

In one embodiment of the invention, a binding method is provided for screening for a compound able to bind selectively to SCN8Asv1.2. The method comprises the steps: providing a SCN8Asv1.2 polypeptide comprising SEQ ID NO 6; providing a sodium channel isoform polypeptide that is not SCN8Asv1.2; contacting the SCN8Asv1.2 polypeptide and the sodium channel isoform polypeptide that is not SCN8Asv1.2 with a test preparation comprising one or more test compounds; and then determining the binding of the test preparation to the SCN8Asv1.2 polypeptide and to the sodium channel isoform polypeptide that is not SCN8Asv1.2, wherein a test preparation that binds to the SCN8Asv1.2 polypeptide, but does not bind to the sodium channel isoform polypeptide that is not SCN8Asv1.2, contains one or more compounds that selectively bind to SCN8Asv1.2.

In another embodiment of the invention, a binding method is provided for screening for a compound able to bind selectively to SCN8Asv2. The method comprises the steps: providing a SCN8Asv2 polypeptide comprising SEQ ID NO 8; providing a sodium channel isoform polypeptide that is not SCN8Asv2; contacting the SCN8Asv2 polypeptide and the sodium channel isoform polypeptide that is not SCN8Asv2 with a test preparation comprising one or more test compounds; and then determining the binding of the test preparation to the SCN8Asv2 polypeptide and to the sodium channel isoform polypeptide that is not SCN8Asv2, wherein a test preparation that binds to the SCN8Asv2 polypeptide, but does not bind to the sodium channel isoform polypeptide that is not SCN8Asv2, contains one or more compounds that selectively bind to SCN8Asv2.

In another embodiment of the invention, a binding method is provided for screening for a compound able to bind selectively to a sodium channel isoform polypeptide that is not SCN8Asv1.1. The method comprises the steps: providing a SCN8Asv1.1 polypeptide comprising SEQ ID NO 4; providing a sodium channel isoform polypeptide that is not SCN8Asv1.1; contacting the SCN8Asv1.1 polypeptide and the sodium channel isoform polypeptide that is not SCN8Asv1.1 with a test preparation comprising one or more test compounds; and then determining the binding of the test preparation to the SCN8Asv1.1 polypeptide and the sodium channel isoform polypeptide that is not SCN8Asv1.1, wherein a test preparation that binds the sodium channel isoform polypeptide that is not SCN8Asv1.1, but does not bind SCN8Asv1.1, contains a compound that selectively binds the sodium channel isoform polypeptide that is not SCN8Asv1.1. Alternatively, the above method can be used to identify compounds that bind selectively to a sodium channel polypeptide that is not SCN8Asv1.2 by performing the method with SCN8Asv1.2 protein comprising SEQ ID NO 6. Alternatively, the above method can be used to identify compounds that bind selectively to a sodium channel isoform polypeptide that is not SCN8Asv2 by performing the method with SCN8Asv2 protein comprising SEQ ID NO 8.

The above-described selective binding assays can also be performed with a polypeptide fragment of SCN8Asv1.1 or SCN8Asv2, wherein the polypeptide fragment comprises at least 10 consecutive amino acids that are coded by a nucleotide sequence that bridges the junction created by the splicing of the 3′ end of exon 2 to the 5′ end of exon 7 in the case of SCN8Asv1.1 or by a nucleotide sequence that comprises nucleotides 618-636 in the case of SCN8Asv2. Similarly, the selective binding assays may also be performed using a polypeptide fragment of a sodium channel isoform polypeptide that is not SCN8Asv1.1 or SCN8Asv2, wherein the polypeptide fragment comprises at least 10 consecutive amino acids that are coded by: a) a nucleotide sequence that is contained within exon 3, 4, 5, or 6, of the SCN8A gene; or b) a nucleotide sequence that bridges the junction created by the splicing of the 3′ end of exon 2 to the 5′ end of exon 3, the splicing of the 3′ end of exon 3 to the 5′ end of exon 4, the splicing of the 3′ end of exon 4 to the 5′ end of exon 5, the splicing of the 3′ end of exon 5 to the 5′ end of exon 6, or the splicing of the 3′ end of exon 6 to the 5′ end of exon 7 of the SCN8A gene.

SCN8A Functional Assays

SCN8A encodes the alpha subunit of a highly conserved voltage gated sodium channel that is implicated in neurological disorders such as ataxia, paralysis, loss of muscle innervation and dystonia. Splice variants of sodium channels may exhibit different voltage gate activity and different binding affinities for compounds, peptides and other small molecules. The identification of SCN8Asv1.1, SCN8Asv1.2, and SCN8Asv2 as splice variants of SCN8A provides a means of screening for compounds that bind to SCN8Asv1.1, SCN8Asv1.2 and/or SCN8Asv2 protein thereby altering the activity or regulation of SCN8A1.1, SCN8A1.2 and/or SCN8Asv2 sodium channels. Assays involving a functional SCN8Asv1.1, SCN8Asv1.2, or SCN8Asv2 polypeptide can be employed for different purposes, such as selecting for compounds active at SCN8Asv1.1, SCN8Asv1.2, or SCN8Asv2; evaluating the ability of a compound to affect the ion channel activity of each respective splice variant; and mapping the activity of different SCN8Asv1.1, SCN8Asv1.2, and SCN8Asv2 regions. SCN8Asv1.1, SCN8Asv1.2, and SCN8Asv2 activity can be measured using different techniques such as: detecting a change in the intracellular conformation of SCN8Asv1.1, SCN8Asv1.2, or SCN8Asv2; detecting a change in the intracellular location of SCN8Asv1.1, SCN8Asv1.2, or SCN8Asv2; or measuring the ion channel activity of SCN8Asv1.1, SCN8Asv1.2, or SCN8Asv2.

Recombinantly expressed SCN8Asv1.1, SCN8Asv1.2, and SCN8Asv2 can be used to facilitate the determination of whether a compound's activity in a cell is dependent upon the presence of SCN8Asv1.1, SCN8Asv1.2, and SCN8Asv2. For example, SCN8Asv1.1, SCN8Asv1.2, and SCN8Asv2 can be expressed by an expression vector in a cell line and used in a co-culture growth assay, such as described in U.S. Pat. No. 6,518,035, to identify compounds that alter the growth of the cell expressing SCN8Asv1.1, SCN8Asv1.2, and SCN8Asv2 from the expression vector as compared to the same cell line but lacking the SCN8Asv1.1, SCN8Asv1.2, and SCN8Asv2 expression vector. Alternatively, determination of whether a compound's activity on a cell is dependent upon the presence of SCN8Asv1.1 can also be done using gene expression profile analysis methods as described, for example, in U.S. Pat. No. 6,324,479. A similar strategy can be used for SCN8Asv1.2, or SCN8Asv2.

Techniques for measuring voltage gated ion channel activity are well known in the art. Methods for expressing sodium channels in Xenopus oocytes and monitoring the activity of these channels, including analyzing the effect of compounds on the activity of sodium channel activity, have been described previously (Dietrich et al., 1998, J. Neurochem. 70(6): 2262-72; Keller et al., 2000, J. Pharmacol. Exp. Ther. 295(1): 367-72; Spampanato et al., 2001, J. Neurosci. 21(19): 7481-7490; Tan et al., 2002, J. Neurosci. 22(13): 5300-5309). The patch clamp technique measures ion current through ion channel proteins and can be used to analyze the effect of drugs on ion channel function. The activity of these channels can be measured electrically (single channel recording) or, alternatively, the patch can be ruptured allowing measurements of the combined channel activity of the entire cell membrane (whole cell recording) (Neher et al., 1978, Pflugers Arch. 375(2): 219-28; Sakman et al., 1984, Annu Rev Physiol. 46: 455-72; Neher et al., 1992, Sci. Am. 266(3): 44-51). Other methods for measuring ion channel activity include optical reading of voltage-sensitive dyes (Cohen et al., 1978, Annual Reviews of Neuroscience 1: 171-82) and extracellular recording of fast events using metal (Thomas et al., 1972, Exp. Cell Res. 74: 61-66) or field effect transistor (From herz et al., 1991, Science 252: 1290-1293) electrodes. High throughput methods for assaying ion channel activity have also been described (see WO 03/006103A2 and U.S. 2002/0028480). A variety of other assays has been used to investigate the properties of sodium channels and therefore would also be applicable to the measurement of SCN8Asv1.1, SCN8Asv1.2, or SCN8Asv2 function.

SCN8Asv1.1, SCN8Asv1.2, or SCN8Asv2 functional assays can be performed using cells expressing SCN8Asv1.1, SCN8Asv1.2, or SCN8Asv2 at a high level. These proteins will be contacted with individual compounds or preparations containing different compounds. A preparation containing different compounds where one or more compounds affect SCN8Asv1.1, SCN8Asv1.2, or SCN8Asv2 in cells over-producing SCN8Asv1.1, SCN8Asv1.2, or SCN8Asv2 as compared to control cells containing an expression vector lacking SCN8Asv1.1, SCN8Asv1.2, or SCN8Asv2 coding sequences, can be divided into smaller groups of compounds to identify the compound(s) affecting SCN8Asv1.1, SCN8Asv1.2, or SCN8Asv2 activity, respectively.

SCN8Asv1.1, SCN8Asv1.2, or SCN8Asv2 functional assays can be performed using recombinantly produced SCN8Asv1.1, SCN8Asv1.2, or SCN8Asv2 present in different environments. Such environments include, for example, cell extracts and purified cell extracts containing the SCN8Asv1.1, SCN8Asv1.2, or SCN8Asv2 expressed from recombinant nucleic acid; and the use of purified SCN8Asv1.1, SCN8Asv1.2, or SCN8Asv2 produced by recombinant means that is introduced into a different environment suitable for measuring ion channel activity.

Modulating SCN8Asv1.1, SCN8Asv1.2 and SCN8Asv2 Expression

SCN8Asv1.1, SCN8Asv1.2, or SCN8Asv2 expression can be modulated as a means for increasing or decreasing SCN8Asv1.1, SCN8Asv1.2, or SCN8Asv2 activity, respectively. Such modulation includes inhibiting the activity of nucleic acids encoding the SCN8A isoform target to reduce SCN8A isoform protein or polypeptide expression, or supplying SCN8A nucleic acids to increase the level of expression of the SCN8A target polypeptide thereby increasing SCN8A activity.

Inhibition of SCN8Asv1.1, SCN8Asv1.2, and SCN8Asv2 Activity

SCN8Asv1.1, SCN8Asv1.2, or SCN8Asv2 nucleic acid activity can be inhibited using nucleic acids recognizing SCN8Asv1.1, SCN8Asv1.2, or SCN8Asv2 nucleic acid and affecting the ability of such nucleic acid to be transcribed or translated. Inhibition of SCN8Asv1.1, SCN8Asv1.2, or SCN8Asv2 nucleic acid activity can be used, for example, in target validation studies.

A preferred target for inhibiting SCN8Asv1.1, SCN8Asv1.2, or SCN8Asv2 is mRNA stability and translation. The ability of SCN8Asv1.1, SCN8Asv1.2, or SCN8Asv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding region of a gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Methods for using RNAi to modify sodium channel activity have been described previously (Keller et al., 2000, J. Pharmacol. Exp. Ther. 295(1): 367-72). Modified and unmodified nucleic acids can be used as anti-sense molecules, RNAi and ribozymes. Different types of modifications can affect certain RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing SCN8Asv1.1, SCN8Asv1.2, and SCN8Asv2 Expression

Nucleic acids encoding for SCN8Asv1.1, SCN8Asv1.2, or SCN8Asv2 can be used, for example, to cause an increase in SCN8A activity or to create a test system (e.g., a transgenic animal) for screening for compounds affecting SCN8Asv1.1, SCN8Asv1.2, or SCN8Asv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of SCN8Asv1 Using Microarrays

To identify variants of the “normal” splicing of the exon regions encoding SCN8A, an exon junction microarray, comprising probes complementary to each splice junction resulting from splicing of the 27 exon coding sequences in SCN8A heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in Van't Veer, et al. (2002 Nature 415:530-536) and Hughes, et al. (2001 Nature Biotechnol. 19:342-7). Inspection of the exon junction microarray hybridization data (not shown) suggested that the structure of at least one junction of SCN8A mRNA was altered in some of the tissues examined, suggesting the presence of SCN8A splice variant mRNA populations. Reverse transcription and polymerase chain reaction (RT-PCR) were then performed using oligonucleotide primers complementary to exons 2 and 7 to confirm the exon junction array results and to allow the sequence structure of the splice variants to be determined.

### Example 2

### Confirmation of SCN8Asv1 and Identification of SCN8Asv2 Using RT-PCR

The structure of SCN8A mRNA in the region corresponding to exons 2 to 7 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 2 and exon 7 of the reference exon coding sequences in SCN8A (NM_{—}014191.1). Based upon the nucleotide sequence of SCN8A mRNA, the SCN8A exon 2 and exon 7 primer set (hereafter SCN8A₂₋₇ primer set) was expected to amplify a 713 base pair amplicon representing the “reference” SCN8A mRNA region. The SCN8A exon 2 forward primer has the sequence: 5′ CTGAGAGCAAGCTCAAGAAACCACCAAA 3′ [SEQ ID NO 14]; and the SCN8A exon 7 reverse primer has the sequence: 5′ CAAGGCAAAAACACT CAGGCAGAACACT 3′ [SEQ ID NO 15].

Twenty-five ng of polyA mRNA from each tissue was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then 72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Clones were then sequenced from each end (using the same primers used for RT-PCR) by Qiagen Genomics, Inc. (Bothell, Wash.).

Sequence analysis of several 713 base pair amplicons expected for normally spliced SCN8A mRNA revealed the existence of an alternative exon 6 (herein referred to as exon 6A) in one of the amplicons. Exon 6A encodes the same number of amino acids as exon 6. However, the amino acid sequence encoded by exon 6A differs from the protein sequence encoded by exon 6 at two amino acid positions (1207V and N212D). This SCN8A isoform mRNA with exon 6A instead of exon 6 is herein referred to as SCN8Asv2 and is represented by SEQ ID NO 3.

At least one different RT-PCR amplicon was obtained from human mRNA samples using the SCN8A₂₋₇ primer set (data not shown). Every human tissue and cell line assayed, except heart, placenta, leukemia promyelocytic (HL-60), salivary gland, lymphoma—Burkitts (Raji), lung—fetal, pancreas, skeletal muscle, trachea, thyroid, bone marrow, ileocecum, adrenal medulla, heart-interventricular septum, and jejunum, exhibited the expected amplicon size of 713 base pairs for normally spliced SCN8A mRNA. In addition, all human tissues and cell lines assayed, except for placenta, leukemia promyelocytic (HL-60), salivary gland, lymphoma—Burkitts (Raji), pancreas, skeletal muscle, thyroid, and bone marrow, also exhibited an amplicon of about 283 base pairs. The tissues in which SCN8Asv1 mRNA was detected are listed in Table 1:

TABLE 1

| Sample | SCN8Asv1 |
|---|---|
| Heart | x |
| Kidney | x |
| Liver | x |
| Brain | x |
| Placenta | |
| Lung | x |
| Fetal Brian | x |
| Leukemia Promyelocytic (HL-60) | |
| Adrenal Gland | x |
| Fetal Liver | x |
| Salivary Gland | |
| Pancreas | |
| Skeletal Muscle | |
| Brain Cerebellum | x |
| Stomach | x |
| Trachea | x |
| Thyroid | |
| Bone Marrow | |
| Brain Amygdala | x |
| Brain Caudate Nucleus | x |
| Brain Corpus Callosum | x |
| Ileocecum | x |
| Lymphoma Burkitt's (Raji) | |
| Spinal Cord | x |
| Lymph Node | x |
| Fetal Kidney | x |
| Uterus | x |
| Spleen | x |
| Brain Thalamus | x |
| Fetal Lung | x |
| Testis | x |
| Melanoma (G361) | x |
| Lung Carcinoma (A549) | x |
| Adrenal Medula, normal | x |
| Brain, Cerebral Cortex, normal; | x |
| Descending Colon, normal | x |
| Prostate | x |
| Duodenum, normal | x |
| Epididymus, normal | x |
| Brain, Hippocamus, normal | x |
| Ileum, normal | x |
| Interventricular Septum, normal | x |
| Jejunum, normal | x |
| Rectum, normal | x |

Sequence analysis of the about 283 base pair amplicon revealed that this amplicon form results from the splicing of exon 2 of the SCN8A hnRNA to exon 7; that is, the coding sequence of exons 3, 4, 5, and 6 is completely absent. Thus, the RT-PCR results confirmed the junction probe microarray data reported in Example 1, which suggested that SCN8A mRNA is composed of a mixed population of molecules wherein in at least one of the SCN8A mRNA splice junctions is altered.

### Example 3

### Cloning of SCN8Asv1 and SCN8Asv2

Microarray, RT-PCR, and sequencing data indicate that in addition to the normal SCN8A reference mRNA sequence, NM_{—}014191.1, encoding SCN8A protein, NP_{—}055006, two novel splice variant forms of SCN8A mRNA also exist in many tissues.

Clones having a nucleotide sequence comprising the splice variants identified in Example 2 (hereafter referred to as SCN8Asv1.1, SCN8Asv1.2, or SCN8Asv2) are isolated using a 5′ “forward” SCN8A primer and a 3′ “reverse” SCN8A primer, to amplify and clone the entire SCN8Asv1.], SCN8Asv1.2, or SCN8Asv2 mRNA coding sequences, respectively. The same 5′ “forward” primer is designed for isolation of full length clones corresponding to the SCN8Asv1.1 and SCN8Asv2 splice variants and has the nucleotide sequence of 5′ ATGGCAGC GCGGCTGCTTGCACCACCA 3′ [SEQ ID NO 16]. The 5′ “forward” SCN8Asv1.2 primer is designed to have the nucleotide sequence of 5′ ATGATCCTGACAGTGTTCTGCCTGAGT 3′ [SEQ ID NO 17]. The same 3′ “reverse” primer is designed for isolation of full length clones corresponding to the SCN8Asv1.2 and SCN8Asv2 splice variants and has the nucleotide sequence of 5′CTAACACTTGGATTCTCTGACCTCTTT 3′ [SEQ ID NO 18]. The 3′ “reverse” SCN8Asv1.1 primer is designed to have the nucleotide sequence of 5′ TCAGGCTTTCTGCGT CAAATAGTATGG 3′ [SEQ ID NO 19].

RT-PCR

The SCN8Asv1.1, SCN8Asv1.2, and SCN8Asv2 cDNA sequences are cloned using a combination of reverse transcription (RT) and polymerase chain reaction (PCR). More specifically, about 25 ng of fetal brain polyA mRNA (BD Biosciences Clontech, Palo alto, CA) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the SCN8A “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted SCN8Asv1.1, SCN8Asv1.2 and SCN8Asv2 mRNAs, products of about 282, 5181 and 5943 bases, respectively. The remainder of the 50 μl PCR reactions from fetal brain is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified SCN8Asv1.1, SCN8Asv1.2, and SCN8Asv2 RT-PCR products from fetal brain are used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative SCN8Asv1.1 clones are identified and prepared for a PCR reaction to confirm the presence of the expected SCN8Asv1.1 exon 2 to exon 7 splice variant structure. A 25 μl PCR reaction is performed as described above (RT-PCR section) to detect the presence of SCN8Asv1.1, except that the reaction includes miniprep DNA from the TOPO TA/SCN8Asv1.1 ligation as a template. An additional 25 μl PCR reaction is performed as described above (RT-PCR section) to detect the presence of SCN8Asv1.2, except that the reaction includes miniprep DNA from the TOPO TA/SCN8Asv1.2 ligation as a template. An additional 25 μl PCR reaction is performed as described above (RT-PCR section) to detect the presence of SCN8Asv2, except that the reaction includes miniprep DNA from the TOPO TA/SCN8Asv2 ligation as a template. About 10 μl of each 25 μl PCR reaction is run on a 1% Agarose gel and the DNA bands generated by the PCR reaction are visualized and photographed on a UV light box to determine which minipreps samples have PCR product of the size predicted for the corresponding SCN8Asv1.1, SCN8Asv1.2, and SCN8Asv2 splice variant mRNAs. Clones having the SCN8Asv1.1 structure are identified based upon amplification of an amplicon band of 282 base pairs, whereas a normal reference SCN8A clone will give rise to an amplicon band of 712 base pairs. DNA sequence analysis of the SCN8Asv1.1 cloned DNAs confirm a polynucleotide sequence representing the deletion of exons 3, 4, 5, and 6. Both the normal reference SCN8A gene and a clone having the SCN8Asv1.2 structure give rise to an amplicon of 5181 base pairs. DNA sequence analysis of the SCN8Asv1.2 cloned DNA confirm a polynucleotide sequence representing SEQ ID NO 5. Both the normal reference SCN8A gene and a clone having the SCN8Asv2 structure give rise to an amplicon of 5943 base pairs. DNA sequence analysis of the SCN8Asv2 cloned DNA confirm a polynucleotide sequence representing SEQ ID NO 7.

The polynucleotide sequence of SCN8Asv1 mRNA contains two open reading frames that encode an amino-terminal and a carboxy-terminal protein, referred to herein as SCN8Asv1.1 and SCN8Asv1.2, respectively. SEQ ID NO 3 encodes the amino terminal SCN8Asv1.1 protein (SEQ ID NO 4). The polynucleotide sequence of SCN8Asv1.1 mRNA (SEQ ID NO 3) lacks a 430 base pair region corresponding to exons 3, 4, 5, and 6 of the full length coding sequence of the reference SCN8A mRNA (NM_{—}014191.1). Deletion of the 430 base pair region results in a protein translation reading frame shift and a premature stop codon in comparison to the reference SCN8A protein reading frame (NP_{—}055006). Therefore the first 92 amino acids of the SCN8Asv1.1 protein are identical to the reference SCN8A (NP_{—}055006), but the next amino acid and the premature stop codon are unique to the SCN8Asv1.1 protein as compared to the reference SCN8A protein (NP_{—}055006). The SCN8Asv1.2 polynucleotide (SEQ ID NO 5) encodes the carboxy terminal SCN8Asv1.2 protein (SEQ ID NO 6). The SCN8Asv1.2 polynucleotide is missing the first 762 base pairs of the SCN8A reference mRNA (NM_{—}014191.1) which encode the first 254 amino acids of the SCN8A protein (NP_{—}055006). The SCN8Asv1.2 polynucleotide is translated in the same frame as the SCN8A reference mRNA (NM_{—}014191.1) and translation is initiated at an AUG codon downstream from the AUG initiation codon utilized by the reference SCN8A protein (NP_{—}055006). The SCN8Asv1.2 protein sequence (SEQ ID NO 6) therefore corresponds to the last 1726 amino acids of the SCN8A reference protein sequence (NP_{—}055006).

The polynucleotide sequence of SCN8Asv2 mRNA (SEQ ID NO 7) contains an open reading frame that encodes a SCN8Asv2 protein (SEQ ID NO 8). SCN8Asv2 mRNA (SEQ ID NO 7) is missing exon 6 as compared to the SCN8A reference sequence (NM_{—}014191.1), but has exon 6A in its place. The protein sequence encoded by exon 6A differs from the protein sequence encoded by exon 6 at only two amino acids positions (I207V and N212D). Thus the SCN8Asv2 protein sequence (SEQ ID NO 8) is different from the SCN8A reference protein sequence (NP_{—}055006) at only two amino acids—I207V and N212D.

### Example 4

### Analyzing Ion Channel Activity of SCN8A and Other Sodium Channel Isoforms

To express SCN8A and other sodium channel isoform channels in Xenopus oocytes, cDNA encoding the appropriate channel is cloned into standard expression vectors, such as pClneo (Promega) or pcDNA3 (Invitrogen), or into a modified pGEM vector (Promega) containing the Xenopus β-globin 5′ and 3′ untranslated region to enhance expression levels as described in Goldin, A. L. (1991, Methods Cell Biol. 36: 487-509). Plasmids are linearized, and RNA is transcribed using the T7 (or SP6) RNA polymerase and the mMessage mMachine kit from Ambion. RNA is either directly injected or diluted prior to injection to obtain maximum current amplitudes of between 0.5 and 5 μA at test potentials 2-7 days after mRNA injection. Current amplitude is measured using a Dagan two-electrode voltage-clamp amplifier and the pCLAMP acquisition software. Leak current is subtracted using the scaled current observed with a P/n protocol. The capacitance and resistance compensation feature on the Dagan is used to minimize the capacitance transient measured at a voltage where Naᵥ channels are not opened. Glass microelectrodes are pulled to achieve resistances of 0.5-1.0MΩ after filling with 1 M KCl measured in the recording solution. Oocytes are prepared by standard techniques and recording is done in ND-96 (98 mM NaCl, 2 mM KCl, 1 mM MgCl₂, 1.5 mM CaCl₂, 5 mM HEPES-Na, pH 7.5). Oocyte membrane potential is held at −80 or −100 mV, and a current-voltage relationship is measured to determine the voltage that produced the maximum inward current. Typically, oocytes are depolarized to 0 mV for 40-100 ms. After a stable peak current is obtained, the indicated concentration of venom, toxin, or compound is added by perfusing the 1 mL recording chamber with ND 96 containing the sample, typically at 2-3 mL/min until stable block is achieved. The oocyte is subsequently perfused with fresh ND-96 to assess reversibility of inhibition.

Cell Culture and HEK Cell Electrophysiology:

For whole-cell voltage-clamp recordings, stably transfected HEK-293 cells or other eukaryotic cell lines expressing SCN8A or other sodium channel isoforms are used. Retro-virus vectors may be used to generate cell lines expressing SCN8A or other sodium channel isoform cell lines. The SCN8A or other sodium channel isoform is cloned in a retroviral expression vector (pLCNX, Clontech). Subsequently virus particles are used to infect HEK-293 cells, and a cell line stably expressing the SCN8A channel is selected. Cells are maintained in either MEM (Minimum Essential Medium) supplemented with 10% fetal bovine serum, 2 mM L-glutamine, 1 unit/mL penicillin/streptomycin or DMEM (Dulbecco's modified Eagles medium) supplemented with 10% fetal bovine serum, mM L-glutamine, 2 units/mL penicillin/streptomycin, 0.02 mg/mL G-418, as appropriate. Cells are plated on poly-D-lysine coated cover slips 16 hours prior to recording and are then washed with mammalian Ringer's solution immediately prior to recording.

Single cell recordings of currents through voltage-activated SCN8A or other sodium channel isoforms are performed at room temperature (20-22° C.) using the whole cell patch-clamp technique (Hamill et al., 1981, Pluegers Archives 391: 85-100). Currents are recorded using a Dagan 3900A or HEKA EPC-9 patch clamp amplifier. Data are stored on a personal computer equipped with HEKA Pulse 8.5 and analyzed using Pulsefit (HEKA Electronics, Lambrecht, Germany), Igor Pro 4.0 (Wavemetrics, Lake Oswego, Oreg.), or Origin 6.0 (Microcal, Northampton, Mass.). Patch pipets are made from borosilicate glass tubing (World Precision Instruments, Sarasota, Fla.), fire-polished, and coated with Sylgard and have a resistance of 1-3MΩ when filled with an internal solution (below) measured in the recording medium. Series resistance and capacitance are compensated using the amplifier's circuitry. Leak resistance is corrected by subtracting the scaled current observed with a P/n protocol. For sodium current measurements, the following solutions are used (in mM): 1) Pipet Solution A: 2 NaCl, 101 Cs gluconate, 20 CsF, 20 CsCl, 11 BAPTA, pH 7.4 adjusted with CsOH; or 2) Pipet Solution B: 140 CsF, 10 NaCl, 1 EGTA, 10 HEPES, pH 7.3 adjusted with CsOH; and 3)Bath Solution A: 15 NaCl, 135 N-methyl-D-glucamine-Cl, 1.8 CaCl₂, 0.5 MgCl₂, pH 7.4 with HEPES; or 4) Bath Solution B: 15 NaCl, 135 choline-Cl, 1 MgCl₂, 10 HEPES, pH 7.3 adjusted with TEA-OH. Purified or synthetic peptides are dissolved in 100 mM KCl and 10 mM HEPES-K, pH 7.5, stored at −20° C., and diluted in the bath solution containing 0.05% BSA (essentially fatty acid free, Sigma, St. Louis, Mo.) to prevent adsorption of the peptide to tubing and perfusion chamber. Data are given as mean±standard error.

VIPR Assay:

Cells expressing SCN8A or other sodium channel isoforms are plated at approximately 100,000 cells/well in poly-D-lysine coated black-wall clear-bottom 96 well plates (Costar # 3667) and are incubated overnight at 37° C. in a 10% CO₂ atmosphere in growth medium. Cells are stained with voltage-sensitive dyes (described in Gonzalez et al., 1997, Chem. Biol. 4: 269-277; Gonzalez et al., 1995, Biophys. J. 69: 1272-1280) by washing twice with 100 μL of Dulbecco's phosphate buffered saline (DPBS) and then incubating in 100 μL of DPBS supplemented with 10 mM glucose, 10 mM HEPES-Na (pH 7.5), and 10 μM CC2-DMPE for 0.7 hours at 27° C. Cells are rinsed twice with 100 μL of Na-free medium (in mM: 160 tetramethylammonium Cl, 0.1 CaCl₂, 1 MgCl₂, 11 glucose, 10 HEPES-K, pH 7.5, [K] approximately 4.5 mM) and then incubated in 100 μL of that medium supplemented with 10 μM DiSBAC₂(3), 20 μM veratridine, 20 nM PbTx-3 (brevetoxin), and test sample at the indicated concentration for 0.7 hours at 27° C. At the end of this incubation, the plate is placed in the VIPR reader, illuminated at 400 nm, and fluorescence emissions at 460 and 580 nm are recorded at 1 Hz. After a 7 second baseline reading, 100 μL of Na solution is added (in mM: 165 NaCl, 4.5 KCl, 2 CaCl₂, 1 MgCl₂, 11 glucose, 10 HEPES-Na, pH 7.5). The change in fluorescence resonance energy transfer (FRET) ratio is recorded as F/F₀ or more explicitly as
F/F₀=((S₄₆₀/S₅₈₀)/(I₄₆₀/I₅₈₀))
where S and I are stimulated and initial fluorescence emissions measurements at the indicated wavelengths. The initial second through seventh readings are averaged for the denominator and the stimulated response is picked as the average of the 12th through the 15th readings. Background fluorescence (˜16-20% of the initial signal) is not subtracted.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid comprising SEQ ID NO 5, or the complement thereof.

2. The purified nucleic acid of claim 1, wherein said nucleic acid comprises a region encoding SEQ ID NO 6.

3. The purified nucleic acid of claim 1, wherein said nucleotide sequence encodes a polypeptide consisting of SEQ ID NO 6.

4. A purified polypeptide comprising SEQ ID NO 6.

5. The polypeptide of claim 4, wherein said polypeptide consists of SEQ ID NO 6.

6. An expression vector comprising a nucleotide sequence encoding SEQ ID NO 6, wherein said nucleotide sequence is transcriptionally coupled to an exogenous promoter.

7. The expression vector of claim 6, wherein said nucleotide sequence encodes a polypeptide consisting of SEQ ID NO 6.

8. The expression vector of claim 6, wherein said nucleotide sequence comprises SEQ ID NO 5.

9. The expression vector of claim 6, wherein said nucleotide sequence consists of SEQ ID NO 5.

10. A method for screening for a compound able to bind to SCN8Asv1.2 comprising the steps of:
(a) expressing a polypeptide comprising SEQ ID NO 6 from recombinant nucleic acid;
(b) providing to said polypeptide a test preparation comprising one or more test compounds; and
(c) measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 6.

15. A method of screening for compounds able to bind selectively to SCN8Asv1.2 comprising the steps of:
(a) providing a SCN8Asv1.2 polypeptide comprising SEQ ID NO 6;
(b) providing one or more sodium channel isoform polypeptides that are not SCN8Asv1.2;
(c) contacting said SCN8Asv1.2 polypeptide and said sodium channel isoform polypeptide that is not SCN8Asv1.2 with a test preparation comprising one or more compounds; and
(d) determining the binding of said test preparation to said SCN8Asv1.2 polypeptide and to said sodium channel isoform polypeptide that is not SCN8Asv1.2, wherein a test preparation which binds to said SCN8Asv1.2 polypeptide, but does not bind to said sodium channel isoform polypeptide that is not SCN8Asv1.2, contains a compound that selectively binds said SCN8Asv1.2 polypeptide.

16. The method of claim 15, wherein said SCN8Asv1.2 polypeptide is obtained by expression of said polypeptide from an expression vector comprising a polynucleotide encoding SEQ ID NO 6.

17. The method of claim 16, wherein said polypeptide consists of SEQ ID NO 6.

18. A method for screening for a compound able to bind to or interact with a SCN8Asv1.2 protein or a fragment thereof comprising the steps of:
(a) expressing a SCN8Asv1.2 polypeptide comprising SEQ ID NO 6 or fragment thereof from a recombinant nucleic acid;
(b) providing to said polypeptide a labeled SCN8A ligand that binds to said polypeptide and a test preparation comprising one or more compounds; and
(c) measuring the effect of said test preparation on binding of said labeled SCN8A ligand to said polypeptide, wherein a test preparation that alters the binding of said labeled SCN8A ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell

21. The method of claim 18, wherein said polypeptide is expressed from an expression vector

22. The method of claim 18, wherein said SCN8Asv1.2 ligand is an SCN8A inhibitor.

23. The method of claim 21, wherein said expression vector comprises SEQ ID NO 5 or a fragment of SEQ ID NO 5.

24. The method of claim 21, wherein said polypeptide comprises SEQ ID NO 6 or a fragment of SEQ ID NO 6.


SEQUENCE LISTING (19 sequences)

SEQ ID NO: 1 - DNA, 40 bp, Homo sapiens
catactatttgacgcagaaagcctgaagacaattgtgggt

SEQ ID NO: 2 - DNA, 40 bp, Homo sapiens
ttcagtgtcatcatgatggcatatgtgacagagtttgtgg

SEQ ID NO: 3 - DNA, 282 bp, Homo sapiens
atggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaagcctga

SEQ ID NO: 4 - protein, 93 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKA

SEQ ID NO: 5 - DNA, 5181 bp, Homo sapiens
atgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacccattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagccaaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttag

SEQ ID NO: 6 - protein, 1726 aa, Homo sapiens
MILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 7 - DNA, 5943 bp, Homo sapiens
atggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggatccatggaactggttagatttcagtgtcatcatgatggcatatgtgacagagtttgtggacctgggcaatgtctcagcgctgagaacattcagggttctccgagctttgaaaactatctctgtaattccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacccattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagccaaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttag

SEQ ID NO: 8 - protein, 1980 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYVTEFVDLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 9 - DNA, 20 bp, Homo sapiens
gacgcagaaagcctgaagac

SEQ ID NO: 10 - DNA, 20 bp, Homo sapiens
tcatgatggcatatgtgaca

SEQ ID NO: 11 - protein, 10 aa, Homo sapiens
FDPYYLTQKA

SEQ ID NO: 12 - protein, 10 aa, Homo sapiens
MILTVFCLSV

SEQ ID NO: 13 - protein, 10 aa, Homo sapiens
AYVTEFVDLG

SEQ ID NO: 14 - DNA, 28 bp, Homo sapiens
ctgagagcaagctcaagaaaccaccaaa

SEQ ID NO: 15 - DNA, 28 bp, Homo sapiens
caaggcaaaaacactcaggcagaacact

SEQ ID NO: 16 - DNA, 27 bp, Homo sapiens
atggcagcgcggctgcttgcaccacca

SEQ ID NO: 17 - DNA, 27 bp, Homo sapiens
atgatcctgacagtgttctgcctgagt

SEQ ID NO: 18 - DNA, 27 bp, Homo sapiens
ctaacacttggattctctgacctcttt

SEQ ID NO: 19 - DNA, 27 bp, Homo sapiens
tcaggctttctgcgtcaaatagtatgg